(19) United States
(12) Patent Application Publication
(10) Pub. No.: US 2004/0073235 A1
(43) Pub. Date: Apr. 15, 2004

(54) Surgical article

(76) Inventors: Robert E. Lund, St. Michael, MN (US); Johann J. Neisz, Coon Rapids, MN (US); Brian P. Watschke, Eden Prairie, MN (US); John W. Westrum JR., Prior Lake, MN (US)

Correspondence Address: Jeffrey J. Hohenshell, AMS Research Corporation, 10700 Bren Road West, Minnetonka, MN, 55343, US

(21) Appl. No.: 10/646,082

(22) Filed: Aug. 22, 2003

Related U.S. Application Data
(63) Continuation-in-part of application No. 09/968,239, filed on Oct. 1, 2001.
(60) Provisional application No. 60/238,771, filed on Oct. 6, 2000.

(30) Foreign Application Priority Data
Oct. 5, 2001 (WO) PCT/US01/31360
Oct. 5, 2001 (EP) 01977580.8-2305
Oct. 5, 2001 (AU) 2001296689
Oct. 5, 2001 (CA) 2425138

(51) Int. Cl. A61B 17/08

(52) U.S. Cl. 606/151

(57) ABSTRACT

A surgical article useful in a sling procedure is disclosed. The surgical article may be used with a sling assembly having a sling, and two end insertion sheaths having a hiatus substantially adjacent a mid portion of the sling.

### CROSS REFERENCE TO RELATED APPLICATIONS

The present application is a continuation-in-part of U.S. patent application Ser. No. 09/968,239, filed Oct. 1, 2001 which claims priority to U.S. Provisional Application Serial No. 60/238,771, filed Oct. 6, 2000 (the entire contents of which are fully incorporated herein by reference).

### BACKGROUND

Urinary incontinence, or the inability to control urination, is a major and debilitating problem affecting millions of people, especially women. The female's natural support system for the urethra is a hammock-like supportive layer composed of endopelvic fascia, the anterior vaginal wall, and a distal attachment to the pubic bone. Weakening and elongation of the pubourethral ligaments and the arcus tendineus fascia pelvis, weakening of the endopelvic fascia and pubourethral prolapse of the anterior vaginal wall are some common characteristics of a patient with urinary incontinence.

Many procedures have been devised to treat urinary incontinence. Some have the goal of elevating the neck of the bladder to return it to a higher retropubic position. Many pubovaginal sling procedures have been developed to treat urinary incontinence. Some of these procedures involve positioning anatomical sling material under the urethra to provide elevation and support of the urethra and/or the bladder neck. Examples of attachment sites for the sling include the anterior or superior portion of the pubis (e.g. with bone anchors and associated sutures), Cooper's ligament, or rectus abdominus fascia. Examples of procedures for treating incontinence are disclosed in U.S. Pat. Nos. 5,112,344; 5,611,515; 5,842,478; 5,860,425; 5,899,909; 6,039,686, 6,042,534 and 6,110,101.

Slings used for pubovaginal procedures differ in the type of implantable material and anchoring methods. In some cases, the sling is placed under the bladder neck and secured via suspension sutures to a point of attachment (e.g. bone) through an abdominal and/or vaginal incision.

Complications associated with procedures for treating incontinence include urinary retention, bladder instability and erosion of an implanted article into surrounding tissue. See Spencer et al, A Comparison of Endoscopic Suspension of the Vesical Neck With Suprapubic Vesicourethropexy for Treatment of Stress Urinary Incontinence, J. Urol. 137: 411, (1987); Araki et al, The Loop Loosening Procedure for Urination Difficulties After Stamey Suspension of the Vesical Neck, J. Urol., 144; (1990); and Webster et al., Voiding Dysfunction Following Cystourethropexy: Its Evaluation and Management, J. Urol., 144 (1990).

With respect to sling procedures, if the sling mesh is too loosely associated with its intended physiological environment, the mesh may be ineffective in supporting the urethra and treating incontinence. Several complications can arise from a mesh that is too tightly placed including retention, sling erosion and other damage to surrounding tissue such as the urethra and vagina.

The TVT Tension-free Vaginal Tape procedure utilizes a knitted Prolene™ nonabsorbable, polypropylene mesh. The mesh is a substantially flat, rectangular woven article. The mesh includes a plurality of holes that are sized to allow tissue ingrowth to help avoid infection. A removable plastic sheath surrounds the mesh and is used during insertion of the mesh. The sling is positioned near the urethra without the use of bone anchors. Once the sheath is removed from the mesh of the TVT product, friction between the mesh and tissue keeps the mesh in position and it becomes very difficult to subsequently adjust the position of the mesh relative to tissue. Attempts to move the sling once the sheath is removed may damage the sling or adjacent tissue such as the urethra or vagina.

The TVT product includes a sling and insertion sheaths that substantially overlap at a mid portion of the sling. The sling is intended to be placed in a U-shaped configuration underneath the patient's urethra. The overlapping portion of the insertion sheaths is substantially adjacent the urethra. This presents a problem when the surgeon attempts to remove the sheaths. After insertion into the body and just prior to removal, the overlapping portion of the insertion sheaths is in the region of greatest curvature of the U-shaped configuration. This tends to cause the insertion sheaths to bind or stick together. This can make removal of the insertion sheaths difficult. Published U.S. patent application Ser. No. 2002/0156487-A1 discloses a clamp placed across overlapping portions of a sling assembly. This arrangement is believed to only exacerbate the problem.

Proper tension of a sling is an important factor for a successful surgical procedure. Surgical approaches to applying tension or slack in a sling procedure vary widely. See Decter, Use of the Fascial Sling for Neurogenic Incontinence: Lessons Learned, The Journal of Urology, Vol. 150, 683-686 (1993). While the TVT procedure suggests using a scissors or hemostat placed between the sling and urethra to set the looseness of the TVT mesh sling, a flat blunt surgical instrument is placed between the sling and urethra in other procedures. See Moir et al., The Gauze-Hammock Operation, The Journal of Obstetrics and Gynaecology of the British Commonwealth, Vol. 75, No. 1 (January 1968) Pps. 1-9.

The TVT sling procedure instructs users to place a scissors or hemostat between the urethra and the sling to ensure ample looseness of the sling. There are several problems associated with this approach. First, the type of scissors or hemostat used to tension the sling may differ in size, potentially causing application of different amounts of looseness for the sling. For example, one surgeon may use a Mayo scissors while another surgeon may use a hemostat or flat, blunt instrument. It is believed that the use of different instruments with different sizes inherently leads to inconsistency in the amount of slack or looseness provided in a sling. This inconsistency could lead to inconsistent therapeutic results, misleading medical data and other clinical errors. Second, even if the same surgical instrument is used, the precise portion of the surgical instrument used to set the looseness of the sling may vary along the length of the instrument. For example, some surgeons use the tips of closed Mayo scissors to tension a sling. Mayo scissors are curved and the precise thickness of a scissors along its length varies significantly. See FIG. 5 of Rackley et al., Tension-free Vaginal Tape and Percutaneous Vaginal Tape Sling Procedures, Techniques in Urology, Vol. 7, No. 2, pp. 90-100 (2001). Depending upon how far the Mayo scissors tips are inserted between the sling and the urethra, the actual amount of looseness provided can vary significantly.

PCT Publication No. WO 03/047435A1 discloses a surgical device for placement against an implant tape. One instrument has an application section with a depression.

Other prior art sling procedures use bone anchors or other methods of securing a sling. A difficulty that contributes to the unnatural positioning of the urethra is that some attachment sites, such as the rectus abdominus fascia or the top of the pubic bone, require very long sutures. Long sutures increase the difficulty in achieving the proper tension in the sutures and sling and increase the chances that intervening anatomical structures may interfere with proper tension. Improper sling tension or sling suture tension can result in increased lateral movement and momentum of the support structures or mesh sling when they are moved due to intra-abdominal pressures.

U.S. Pat. No. 5,863,315 discloses a method of tensioning a suspended tissue mass. The method utilizes a suture tensioner comprising a handle, a main body and an annular recess.

More than a year prior to the filing date of the present application, Vesica Sling Kits were sold (by Boston Scientific, Microvasive, USA) in the United States that included a Suture Spacer. Surgeons were instructed to place the Suture Spacer on the top of the pubic tubercle (which is a location remote from the sling and remote from the urethra, vagina and bladder neck). The surgeon then placed a suture about the Suture Spacer and tied a knot. As the knot was tied, the Suture Spacer was pulled downward onto the top of the pubic bone. Six or seven additional throws were tied and the Suture Spacer was withdrawn.

U.S. Pat. No. 6,050,937 discloses a tension/pressure monitor. The device is said to measure and indicate the degree of tension in a surgical sling.

U.S. Pat. No. 5,474,518 discloses a device for correcting urinary incontinence by use of vesical suspension. The device includes a box that houses a drum with a toothed wheel that engages a worm gear.

U.S. Pat. Nos. 4,938,760 and 4,969,892 disclose a method of suspending the urethrovesical junction in females. An anchoring means for anchoring a suture in tissue is disclosed. The anchoring means comprises a rotating spool, a driving gear and an adjusting means.

PCT International Pub. No. WO 01/39670 discloses an implantable support sheet for providing suburethral stabilization for female patients. A clip is disclosed that inhibits folding of a central part of the sheet about its longitudinal axis.

U.S. Pat. No. 6,106,545 discloses a suture tensioning and fixation device for attachment of tendon to muscle or reattachment of ligaments to bone. The device includes a retaining element and suture thread engaging portions.

U.S. Pat. No. 6,117,067 discloses a device for the height-adjustable fixing and support of internal organs. The device includes a sling, threads, tube, small capsule and chamber. A needle is used to introduce or extract liquid.

U.S. Pat. No. 6,068,591 discloses an apparatus for treatment of female stress urinary incontinence with a support harness. The patent discloses an adjustable setting and Carter pin.

### BRIEF SUMMARY

The present invention is directed to an article useful in surgical procedures. The article assists surgeons in providing consistent, repeatable relationships between implantable materials such as slings, and target tissue such as urethra tissue. In use with a plurality of different surgeons and a plurality of different patients, the present invention can contribute to consistent, repeatable medical results, more reliable medical data and improved medical decisions.

The article comprises a portion adapted to be grasped, and at least one tensioning member that is sized and shaped to afford predetermined looseness of an anatomical support material, such as a sling, relative to anatomical tissue, such as a urethra.

A variety of procedures are contemplated, including, for example, pubovaginal sling procedures. The present invention is particularly suitable for use in a sling procedure that places a sling in a therapeutically effective position. Preferably, the sling is placed to control the pressure applied to the urethra to obtain or restore normal anatomy and continence.

In a preferred embodiment, the article comprises a plurality of tensioning members. In one embodiment the article includes movement means for moving at least one tensioning member between an open position for receiving the anatomical support material and a closed position that associates the article with the anatomical support material. Preferably, the movement means comprises means for affording substantial parallel movement between tensioning members to resist wrinkling of the anatomical support material.

In another preferred embodiment, the plurality of tensioning members are arranged to afford a plurality of tortuous paths of different lengths, and the anatomical support material may be associated with the article along one of the tortuous paths.

In another embodiment, the article includes means for moving at least one of the tensioning members relative to another tensioning member to change the length of a tortuous path.

In another aspect, the present invention comprises a device for applying tension to a surgical sling.

In another aspect, the invention comprises a tensioning device comprising a base member having a plurality of tensioning members extending outwardly from the base member, thereby creating a tortuous pathway for sling material positioned thereon. When the sling material is used in a pubovaginal sling procedure, the tensioning device provides additional tensioning support to the urethral body, the bladder neck, or both.

The tensioning device of the present invention may have a number of different configurations. For example, in one embodiment, the base member is arcuate. In an alternate embodiment, the base member is rectangular. Additionally, the base member may include a directional indicator to aid the user in applying the device. In yet another embodiment, the base member may include an integral or detachable grasping member. The base member may further include at least one sling material locking member having an open position which allows removal of the sling material from the device, and a closed position to the retain sling material within the device.

The tensioning members of the present invention may comprise a plurality of configurations, including circular members, oval members, or other shaped members capable of retaining material disposed therein. Similarly, the tensioning members may be manufactured from a plurality of materials including, without limitation, silicone elastomer, acetate, acetal polyurethane, acrylic, elastomer, stainless steel, polysulfone, nylon, polycarbonate, polyethermide, acetal, ABS, bioresorbable materials or other biologically-compatible materials for temporary or permanent implantation.

In another form, the device remains implanted and can be adjusted in the subsequent post operative period by removing all or some of the tensioning members or by constructing the tensioning members of a bioresorbable material.

Additionally, the tensioning members may be constructed so that they indicate the preload on the sling through a deflection or alignment of the tensioning members.

The present invention may further include various methods of using the tensioning device for creating and using a tortuous pathway to apply tensioning force to a material disposed therein.

In another aspect, the present invention comprises a method of providing a uniform distance between the urethra and a sling comprising: providing a sling and tensioning article, associating the tensioning article with the sling, implanting the sling and associated tensioning article in a position substantially adjacent or, alternatively, just touching the urethra, and then removing the tensioning article. Optionally, the step of providing a sling and tensioning article includes the step of providing an insertion sheath surrounding the sling; and the step of associating the tensioning article with the sling includes the step of associating the sling and sheath combination with the tensioning article.

In another aspect, the present invention comprises a surgical article for associating with a sling assembly or sling. The sling assembly preferably comprises a sling having first and second end portions and a mid portion, a first insertion sheath substantially enclosing the first end portion of the sling, and a second insertion sheath substantially enclosing the second end portion of the sling. The first and second insertion sheaths preferably have ends that are spaced apart so that the mid portion of the sling is free of any insertion sheath. During use, the sling assembly is free of any overlapping sheath portions underneath the urethra.

The surgical article comprises a first jaw and a second jaw. The first and second jaws are movable between an open position to receive the sling assembly or sling therebetween, and a closed position for associating the surgical article with the sling assembly or sling. The first jaw has a predetermined size and shape for assisting a surgeon in implanting the sling with appropriate tension. Preferably, the first jaw has a width and the width is greater than the space between the ends of the first and second insertion sheaths.

The jaws may be any suitable size and shape including flat and arcuate shapes. The first and second jaws may have sling assembly engagement surfaces, or insertion sheath engagement surfaces. Alternatively, the jaws may be designed to engage portions of both the insertion sheaths and the sling.

Preferably, the surgical article includes biasing means for biasing the first and second jaws toward the closed position, and manually engageable handles, operatively associated with the first and second jaws, for manually moving the jaws from the closed position toward the open position against the bias of the biasing means.

In one embodiment, the first and/or second jaws include a slot. In another embodiment, the jaws include appendages projecting therefrom. The appendages may be used to hold portions of the sling assembly (e.g. the sling or the insertion sheaths or both) in a predetermined orientation. In a preferred embodiment, the appendages are flexible so that they can deflect. In another embodiment, a thin material is situated between the appendages. The appendages and thin materials may be adapted to substantially engage the mid-portion of the sling.

In another aspect, the present invention comprises the combination of a sling assembly and surgical spacer article.

In another aspect, the present invention comprises a method of treating incontinence comprising the steps of: (i) providing a support material with at least a portion that is elastically deformable, (ii) elastically deforming at least a portion of the support material to tension the support material; (iii) providing a tensioning article, (iv) applying the tensioning article to the tensioned support material to retain at least a portion of the support material in an elastically deformed condition; (v) implanting the support material with applied tensioning article in a patient; and vi) removing the tensioning article to increase the tension provided by the support material.

In another method, the present invention comprises a method of reducing the looseness of an implanted sling comprising the steps of: providing a tensioning article, and associating the tensioning article with the sling in vivo to tighten the sling.

In another method, the tensioning member remains implanted and provides an elastic stress relieving component to the sling in the immediate or long term post operative period. This is particularly useful when the sling is made of an elastic material such as a silicone elastomer.

In another aspect, the present invention comprises a method of treating a patient's urological disorder. The method includes the steps of i) inserting a sling assembly comprising a sling with a mid-portion, the sling having insertion sheaths enclosing end portions thereof, the insertion sheaths having ends proximate the mid-portion of the sling that are spaced apart, and a spacer article associated with the sling assembly or sling; ii) verifying that the spacer article is in at least close proximity to the patient's urethra; iii) removing the spacer article; and iv) removing the insertion sheaths. In one embodiment, the step of removing the spacer occurs before the step of removing the insertion sheaths. In another embodiment, the step of removing the insertion sheaths occurs before the step of removing the spacer. Preferably, the step of inserting the sling assembly includes the step of making a vaginal incision, and the step of removing the spacer article includes the step of removing the spacer article through the vaginal incision. Various different surgical approaches may be utilized.

In another aspect, the present invention comprises a kit for treating incontinence. The kit comprises surgical articles for implanting a surgical sling, and a tensioning article.

Other features and advantages of the present invention will become apparent from a consideration of the following detailed description.

### BRIEF DESCRIPTION OF THE DRAWINGS

Other features and advantages of the present invention will be seen as the following description of particular embodiments progresses in conjunction with the drawings, in which:

FIG. 1 shows a perspective view of an embodiment of tensioning device of the present invention;

FIG. 2a is a perspective view of the tensioning device of FIG. 1 engaging sling material positioned thereon;

FIG. 2b is a perspective view of an alternate embodiment of the tensioning device of the present invention engaging sling material positioned therein;

FIG. 3 shows a perspective view of an embodiment of tensioning device of the present invention having an orientation indicator positioned thereon;

FIG. 4a shows a perspective view of another embodiment of tensioning device of the present invention having a grasping structure disposed on the base member;

FIG. 4b shows a perspective view of an alternate embodiment of the tensioning device of the present invention having a grasping member disposing a directional indicator thereon positioned on the base member;

FIG. 5a shows a perspective view of another embodiment of tensioning device of the present invention having a locking member positioned thereon;

FIG. 5b shows a perspective view of an alternate embodiment of the tensioning device of the present invention having another embodiment of locking member positioned thereon;

FIG. 6 shows a perspective view of an alternate embodiment of the tension device of the present invention wherein at least one tensioning member is oval;

FIG. 7 is another embodiment of tensioning device according to the present invention that includes a securement member;

FIGS. 8a-8d are views of an alternative embodiment of the present invention wherein:

FIG. 8a shows an end view of an alternative embodiment of tensioning device shown in a closed position;

FIG. 8b shows a side view of the device of FIG. 8a;

FIG. 8c is an end view of the device of FIG. 8a shown in an open position;

FIG. 8d is a side view of the device of FIG. 8c;

FIGS. 9a-9d are views of an alternative embodiment of the present invention wherein:

FIG. 9a shows an end view of an alternative embodiment of tensioning device shown in a closed position;

FIG. 9b shows a side view of the device of FIG. 9a;

FIG. 9c is an end view of the device of FIG. 9a shown in an open position;

FIG. 9d is a side view of the device of FIG. 9c;

FIGS. 10a-10d are views of an alternative embodiment of the present invention wherein:

FIG. 10a shows an end view of an alternative embodiment of tensioning device shown in an open position;

FIG. 10b shows a side view of the device of FIG. 10a;

FIG. 10c is an end view of the device of FIG. 10a shown in a closed position;

FIG. 10d is a side view of the device of FIG. 10c;

FIG. 11a is an end view of another embodiment of tensioning article and sling according to the present invention with tensioning indicia located thereon, which tensioning article is shown in an open position;

FIG. 11b illustrates the assembly of FIG. 11a in a closed position;

FIG. 12 is a perspective view of another embodiment according to the present invention which embodiment includes an adjustment feature;

FIG. 13 is a perspective view of another embodiment of tensioning device according to the present invention;

FIG. 14 is an end view of the article of FIG. 13 with a sling threaded through the article in a first orientation;

FIG. 15 is another end view of the article of FIG. 13 with a sling threaded through the article in a second orientation;

FIG. 16a is a side view of another embodiment of surgical article according to the present invention;

FIG. 16b is an end view of the article of FIG. 16a;

FIG. 17a is a side view of another embodiment of tensioning device according to the present invention;

FIG. 17b is a side view of the tensioning device of FIG. 17a with a sling inserted therebetween;

FIG. 18a is a side view of another embodiment of tensioning device according to the present invention shown in a closed position;

FIG. 18b is a side view of another embodiment of tensioning device according to the present invention shown in an open position;

FIG. 18c is a view of the tensioning device of FIGS. 18a and 18b with a sling placed therein;

FIG. 19 is a flow chart showing a surgical procedure according to the present invention;

FIG. 20 is a schematic view of a tensioning article, sling and selected portions of the anatomy of a patient, which view illustrates removal of the tensioning article after it sets sling tension;

FIG. 21 is a schematic view of a tensioning article, sling and selected portions of the anatomy of a patient, which view illustrates implantation of the tensioning article to increase the tension of the sling;

FIG. 22 is a flowchart showing an example of a method according to the present invention;

FIG. 23 is a schematic illustration of overlapping sheath components of a sling assembly;

FIG. 24 is a perspective view of another embodiment of surgical article according to the present invention;

FIG. 25 is a perspective view of the surgical article of FIG. 24 with jaws shown in an open position to receive a sling assembly;

FIG. 26 is a perspective view of the surgical article and sling assembly of FIG. 25 with the jaws shown in a closed position

FIG. 27 is a perspective view of another embodiment of surgical article according to the present invention;

FIG. 28 is a perspective view of another embodiment of surgical article according to the present invention;

FIG. 29 is a perspective view of another embodiment of surgical article according to the present invention;

FIG. 30 is a perspective view of another embodiment of surgical article according to the present invention, shown with jaws in an open position;

FIG. 31 is a perspective view of the embodiment of FIG. 30 with jaws in an open position to receive a sling assembly;

FIG. 32 is a perspective view of another embodiment of surgical article according to the present invention;

FIG. 33 is another perspective view of the components of FIG. 31 with the jaws of the surgical article in a closed position; and

FIG. 34 is another perspective view of the surgical article of FIG. 32 with jaws in a closed position and a portion of a sling assembly received therebetween.

### DETAILED DESCRIPTION

Disclosed herein is a detailed description of various illustrated embodiments of the invention. This description is not to be taken in a limiting sense, but is made merely for the purpose of illustrating the general principles of the invention. The section titles and overall organization of the present detailed description are for the purpose of convenience only and are not intended to limit the present invention.

The present invention comprises an article, preferably a tension control article, for use in conjunction with an implantable article such as a sling. In a preferred embodiment, the tension control article is utilized in conjunction with a sling for treating urinary incontinence. The tension control article may be used in conjunction with a wide variety of slings and other surgical procedures. For example, the present invention may be utilized in conjunction with the slings and procedures described in U.S. Pat. Nos. 5,520,700; 5,611,515; 5,842,478; 5,860,425; 5,972,000; 6,039,686, 6,042,534, 6,110,101, and 6,612,977; and published U.S. patent application Ser. Nos. 2002-0107430-A1, 2002-0147382, 2002-0099258-A1, 2003/0130670-A1 and U.S. Ser. No. 2002-0099259-A1. Commercial examples of slings, instructions for use and kits that may be modified to incorporate the present invention include the In-Fast Sling System and the SPARC Sling System and the Monarc Sling System available from American Medical Systems of Minnetonka, Minn., and the transvaginal TVT Sling System available from Ethicon (a division of Johnson & Johnson).

Preferably, the tension control article is associated with an implantable material (e.g. a sling). Suitable implantable materials associated with the present invention include synthetic and non-synthetic materials. Suitable non-synthetic implantable materials include human fascia lata, treated animal (e.g. bovine or porcine or equine pericardium) tissue, autologous tissue, cadaver tissue, homografts, xenografts, heterografts, allografts and combinations of such materials. Suitable synthetic materials include knitted polypropylene slings alone, such slings with surrounding sheaths, or silicone coated polymer slings, such as those described in published U.S. patent application Ser. No. 2002/0072694-A1. Alternatively, the tension control article may be associated with sutures associated with slings. Such sutures typically extend from an implanted bone anchor on the pubic bone, or from the rectus abdominus fascia. These sutures hold the sling in place in the body.

The present invention may also be used in conjunction with surgical procedures other than those designed to strictly address incontinence. For example, the present invention may be used in conjunction with a sacral colpopexy procedure designed to treat vaginal prolapse.

The tension control article of the present invention, when used with transvaginal or suprapubic surgical anatomical support material (e.g. a sling) or sutures, is designed to provide an adjustable tensioning or spacing mechanism as an objective aid for surgeons in associating the sling or suture with a therapeutically effective position. The article of the present invention assists surgeons in consistently and repeatably associating a sling with its intended physiological environment (e.g. the bladder neck or urethra, or both).

The tension control article is preferably positioned on a portion of anatomical support material. In one embodiment, the tension control article has a plurality of tensioning members that are sized and shaped to provide a tortuous pathway for the sling material. When the tension control article is associated with the sling material and the sling material is placed at its intended anatomical location, the tension control article results in an increase in the supportive tension that is applied by the sling to anatomical structures relative to that supportive tension that would be applied to the anatomical structures in the absence of the tension control article.

In one embodiment, the size and shape of the tensioning members are selected to provide a predetermined slack in the sling material once the article is removed from the sling material. For example, for a tension free surgical sling procedure for treating incontinence, the tension control article may be associated with the sling and the sling/tension control article combination may be implanted to just touch the urethra of a patient. In this example, once this penultimate orientation of the sling and urethra is achieved, the tension control article may then be removed to ensure a consistent, uniform amount of slack is provided between the sling and the urethra. Providing a uniform, consistent, repeatable amount of looseness in each surgical procedure reduces the chances that patient data is corrupted by the vagaries associated with a particular surgeon's preferences or lack of training or experience. As a result, it is believed that the present invention can lead to more consistent medical results.

The tension control article of the present invention may be constructed of a wide variety of materials. Suitable materials include those that may be permanently implanted in the body, temporarily implanted, and/or completely removed prior to the end of the surgical procedure. The material used to construct the tension control article should be biocompatible and may comprise bioresorbable materials or permanent, biocompatible materials or combinations thereof.

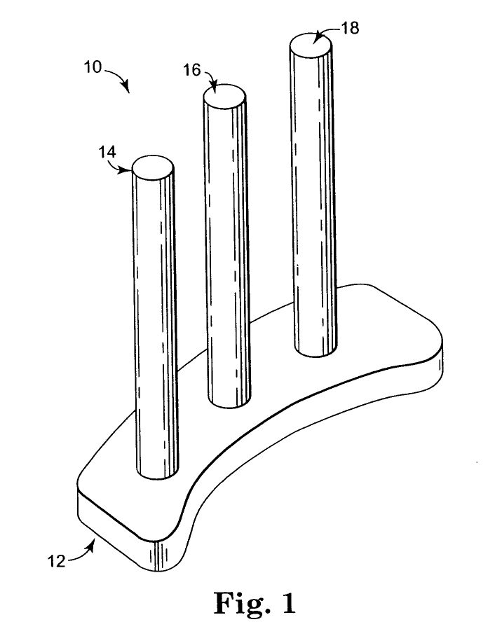

FIG. 1 shows an embodiment of the anatomical support adjustment and tension control article 10 for use with suspension sutures, surgical slings, or other anatomical supports. In this embodiment, the present invention includes a base member 12 and three tensioning members 14, 16, and 18 attached thereto. Optionally, more than three tensioning members may be positioned on the base member. The base member 12 is preferably arcuate and includes beveled edges to reduce or eliminate damage to the surrounding tissue and anatomical support material disposed thereon. In an alternate embodiment, the body member 12 may form any other configuration which facilitates support of the urethra and which minimizes damage to the surrounding tissue and anatomical structures. The device 10 may be manufactured in a plurality of sizes to accommodate the physiological or anatomical constraints of the patient and the location of use. The configuration of the device 10 enables a user to adjust the length of anatomical support material positioned therein and adjustably control the supportive tension applied to tissue.

The device 10 may be constructed of a plurality of materials, including, for example, titanium, stainless steel, nylon, polycarbonate, polysulfone, ABS, ultem, polyetherimide, and polyacetate or combinations thereof, thereby providing a relatively rigid device. In an alternate embodiment of the present invention, the device 10 may be manufactured from moderately flexible materials, such as acetal, or soft flexible materials, such as silicon elastomer or polyurethane, should a more flexible support mechanism be desired. In yet another embodiment, the device 10 may be manufactured from biodegradable materials or polymers. The device 10 may further include or be manufactured from materials having distinct radio opacities or echogenic properties, thereby enabling location of the device 10 in post-surgical procedures. In yet another embodiment, the present invention may be manufactured from materials having distinct optical properties, wherein the application of force to device 10 alters the visual appearance of, or light transmission through, the device 10. Furthermore, it is considered within the scope of the claimed invention to construct the device 10 from multiple materials. For example, the device 10 may comprise a base member 12 constructed of polyacetate, or a similar rigid material, and the tensioning members 14, 16, and 18, respectively, constructed of a flexible material. Other biocompatible materials and material combinations not specifically listed herein, may also be used to fabricate the device 10 and are included within the scope of the claimed invention.

The members 14, 16 and 18 may be integrally molded with the base portion. Alternatively, they could be releasably attached to the base portion to afford adjustment of the sling. For example, the members 14, 16 and 18 may be constructed to be separable from the base portion by use of a remotely actuated device (e.g. a device that utilizes electromagnetic energy). In particular, a magnetic attachment of one or more of the members 14, 16 and 18 and the base portion 12 may be provided. This magnetic attachment may be eliminated by a remotely activated device. This embodiment affords adjustment in the tension of a sling post operatively without requiring a subsequent incision.

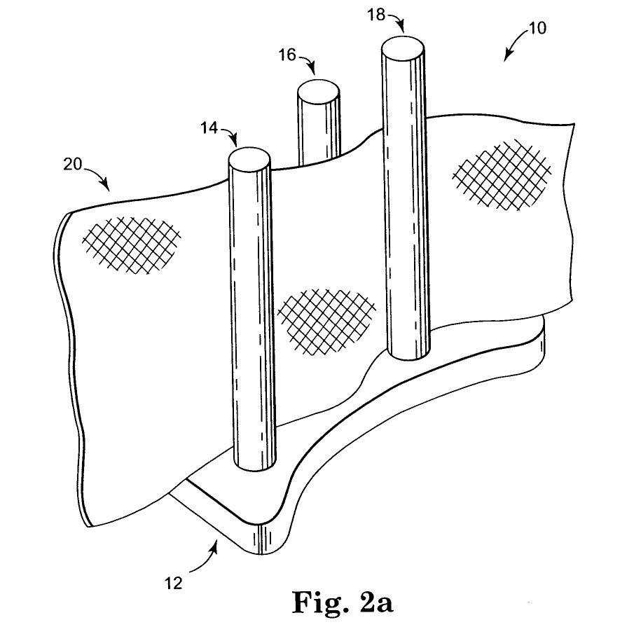

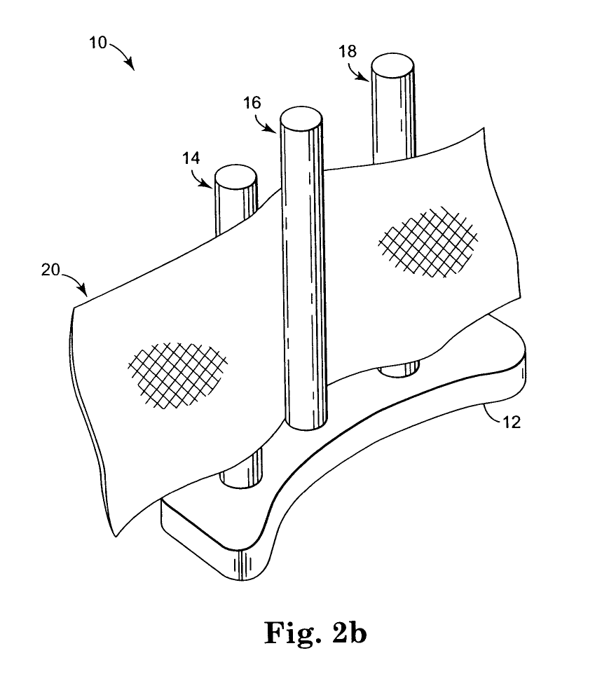

FIGS. 2a and 2b show alternative methods of positioning the present invention on a portion of anatomical support material. FIG. 2a shows one method of using the tensioning device 10 to engage a portion of anatomical support material 20, wherein the material 20 is positioned within a tortuous pathway formed by the plurality of tensioning members 14, 16, and 18 respectively. FIG. 2b shows an another method of using the tensioning device 10, wherein the anatomical support material 20 traverses an alternate tortuous pathway formed by the plurality of tensioning members 14, 16, and 18.

Generally, the longer the tortuous path, the greater the slack provided in the sling 20 once the tension control article 10 is removed. Also, the longer the tortuous path, the more slack is taken up in a sling 20 once the tension control article 10 is associated with the sling. For the same clip 10, the length of the tortuous path in FIG. 2a is different than the length of the tortuous path in FIG. 2b. As a result, the same tension control article may be utilized to provide a plurality of different slacks in the anatomical support material (e.g. sling) 20.

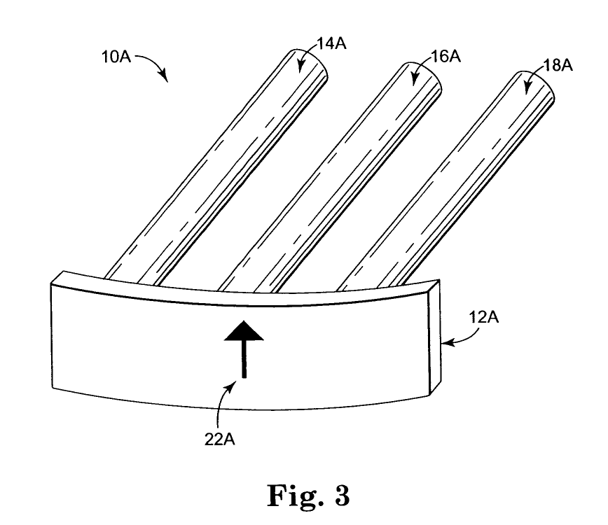

A second embodiment of tension control article 10A is illustrated in FIG. 3. The tension control article 10A has tensioning members 14A, 16A and 18A, and base member 12A. The tension control article 10A further comprises a directional indicator 22A included on the body member 12A. The directional indicator 22A assists the user in properly applying the device 10A to a portion of anatomical support material. As shown in FIG. 3, the directional indicator 22A may comprise an arrow printed on, embossed or integrally disposed on a surface of the base member 12A. Alternatively, the directional indicator may include figures, shapes, letters, or other markings formed, printed, or otherwise included on the device 10A. In another embodiment, the base member 12A may include a tension scale, enabling the user to determine the amount of load imposed on the tensioning members. Alternatively, displacement of flexible members may be used to indicate the applied load. For example, deflection or alignment of the tensioning members may be used to indicate the relative preload on the sling 20.

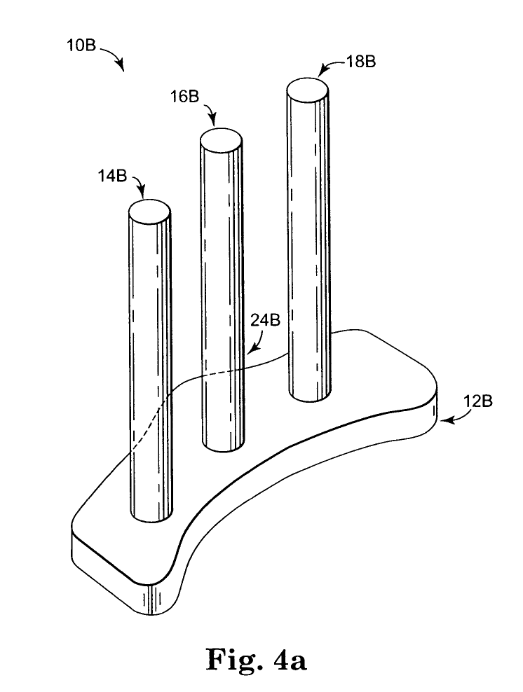

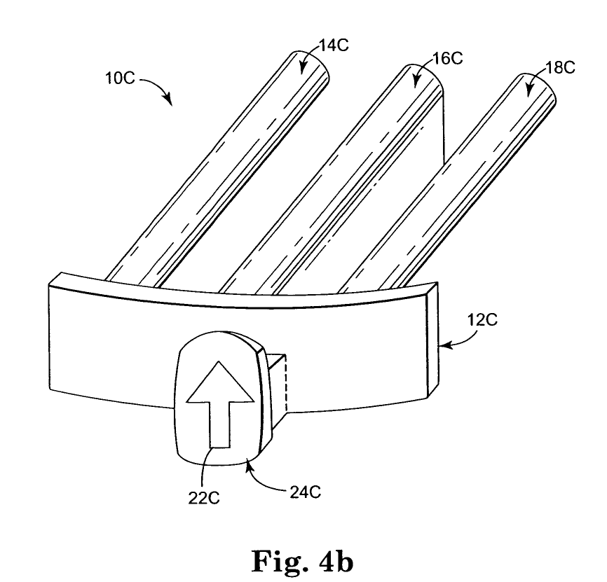

FIGS. 4a and 4b show additional embodiments of the present invention. Tension control article 10B has tensioning members 14B, 16B and 18B, and base member 12B. Tension control article 10C has tensioning members 14C, 16C and 18C, and base member 12C. As shown in FIG. 4b, the device 10C may include a grasping member 24C disposed on or attachable to the base member 12C. In FIG. 4a, the grasping member 24B is integral with the base member and comprises an arcuate discontinuity in the base member 12B. Optionally, the free (unattached) ends of tensioning members 14, 16 and 18 could include an enlarged portion or ledge that retains the sling material or sutures in place.

The grasping member 24C aids the user in applying, positioning, and removing the device 10C from the anatomical support material 20C. FIG. 4b shows the grasping member 24C further comprising a directional indicator 22C positioned thereon, thereby aiding the user in applying the device 10C. In an alternative embodiment of the present invention, a detachable grasping member 24C is contemplated.

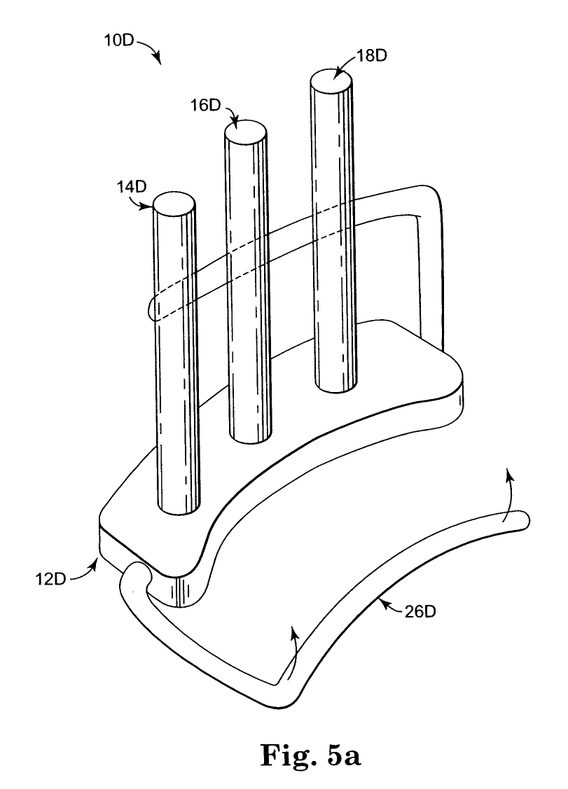

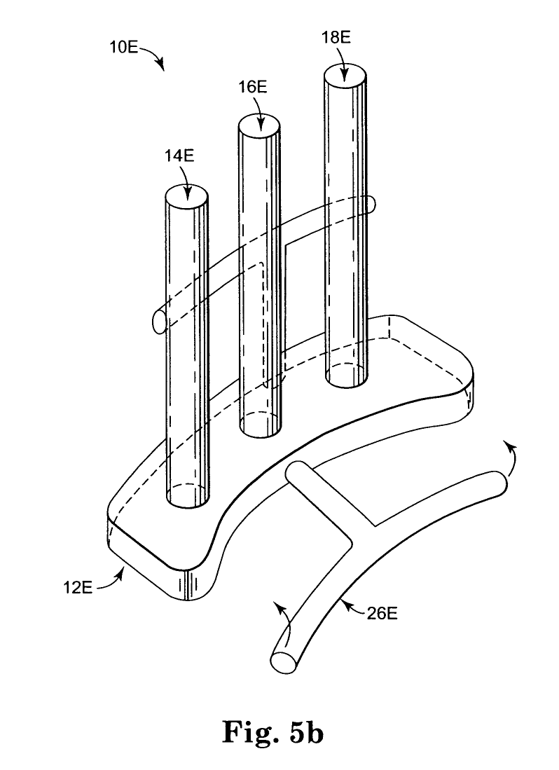

Additional embodiments of the present invention are shown in FIGS. 5a and 5b. Tension control article 10D has tensioning members 14D, 16D and 18D, and base member 12D. Tension control article 10E has tensioning members 14E, 16E and 18E, and base member 12E. Base member 12D of the device 10D includes at least one material locking member 26D. The locking member 26D has an open position permitting the movement of anatomical support material 20 (see FIG. 2a) between the tensioning members 14D, 16D, and 18D, respectively, and a closed position restricting the movement of the anatomical support material 20 relative to the tension control article 10D. The at least one locking member may be manufactured from a plurality of materials having sufficient structural rigidity to prevent material movement, thereby preventing accidental or unintentional adjustment of the tension applied by the anatomical support material. The locking member 26E of FIG. 5b is located on the sides of the base portion 12E, as opposed to the ends (see FIG. 5a).

In another embodiment, the article of the present invention may include a spring biased locking member that is biased toward the closed position. In the closed position, the locking position retains a portion of the sling 20 in a pre-tensioned, elastically deformed condition. Placing the pretensioned sling and associated article in the patient and then subsequently removing the association between the article and the pre-tensioned sling can result in an increase in the tension encountered by a target anatomical structure such a urethra.

The tensioning members 14, 16, and 18 (and those to which a letter suffix has been added herein) may be manufactured from a plurality of materials. For example, the tensioning members 14, 16, and 18 may be manufactured from a flexible material, thereby providing a dynamic tensioning device capable of absorbing temporary variations in supportive loading. If desired, in an alternative embodiment the tensioning members 14, 16, and 18 may be manufactured from a rigid material, permitting the operator to forcibly remove a tensioning member if desired, thereby resulting in decreased support tension applied by the anatomical support material. In an alternate embodiment, the tensioning member may be manufactured from a pliable material, thereby permitting the user to easily position and apply the device 10.

The spacing and number of tensioning members may be adapted to adjust the tension of the anatomical support material disposed on the device 10. For example, a greater number of tensioning members would provide a more tortuous pathway, resulting in greater anatomical support tension or spacing adjustment. The exterior of the tensioning members are preferably smooth. In another embodiment of the present invention, the exterior of at least one of the plurality of tensioning members may be textured to increase anatomical support material retention, or to increase stability within the body if the tension control article is permanently implanted, or to promote tissue ingrowth.

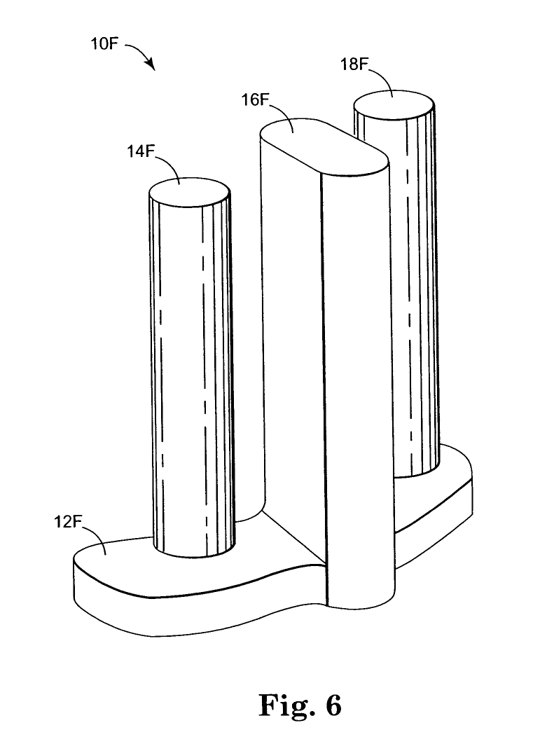

FIG. 6 shows another embodiment of tension control article 10F including tensioning components 14F, 16F, and 18F. In FIG. 6, tensioning member 16F is oval. The tensioning members 14F, 16F, and 18F may be formed in a plurality of shapes and combinations thereof, including, without limitation, triangular, rectangular, oval, hexagonal, octagonal and diamond.

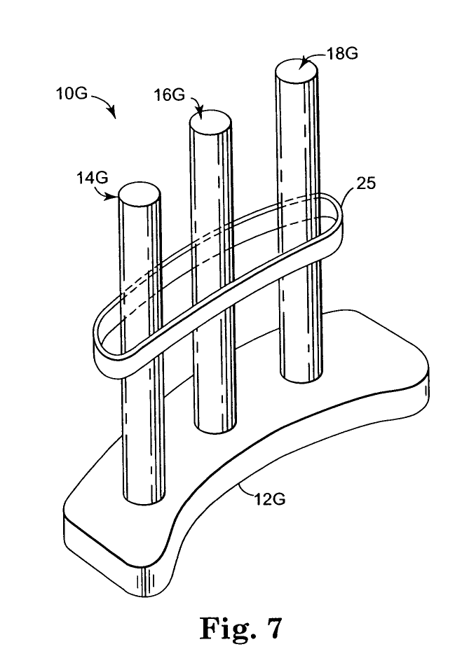

FIG. 7 illustrates another embodiment of tension control article 10G. Tension control article 10G has tensioning members 14G, 16G and 18G, and base member 12G. A band 25 may also be used in conjunction with tension control article 10G to retain the association between the sling 20 and tension control article 10G. The band 25 is placed on the tension control article 10G after the sling is associated with the tension control article 10G so that the band 25 prevents or blocks separation of the tension control article 10G from the support material 20.

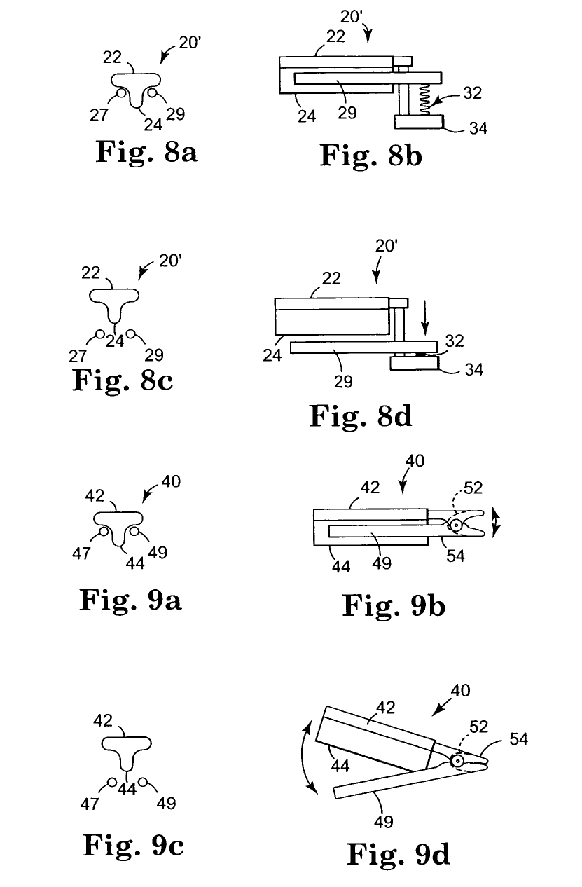

FIGS. 8a-8d show another embodiment of tension control article 20′ according to the present invention. The tension control article 20′ comprises a base portion 22 with integral tensioning member 24, handle 34, and movable tensioning members 27 and 29.

The tension control article 20′ is movable between an open position (FIGS. 8c and 8d) with the tensioning members 27 and 29 spaced from base portion 22 so that the tension control article 20′ may readily receive a sling, and a closed position (FIGS. 8a and 8b) with the tensioning members 27, 29 closer to the base portion 22 than in the open position.

A spring 32 biases the tension control article 20′ toward the closed position. Manual pressure on handle 34 moves the tension control article from the closed toward the open position. The handle 34 is preferably designed so that major surfaces of the base portion 22 and tensioning members 27, 29 remain substantially parallel between the open and closed positions. Substantial parallel movement resists binding or wrinkling of the sling when the tension members 27 and 29 clamp onto the sling.

FIGS. 9a-9d show another embodiment of tension control article 40 according to the present invention. The tension control article 40 comprises a base portion 42 with integral tensioning member 44, handle 54, and movable tensioning members 47 and 49.

The tension control article 40 is movable between an open position (FIGS. 9c and 9d) with the tensioning members 47 and 49 spaced from base portion 42 so that the tension control article 40 may readily receive a sling, and a closed position (FIGS. 9a and 9b) with the tensioning members 47, 49 closer to the base portion 42 than in the open position.

A spring 52 biases the tension control article 40 toward the closed position. Manual pressure on handle 54 moves the tension control article from the closed toward the open position. The movement between the open and closed positions is pivotal movement about a point on or substantially adjacent handle 54.

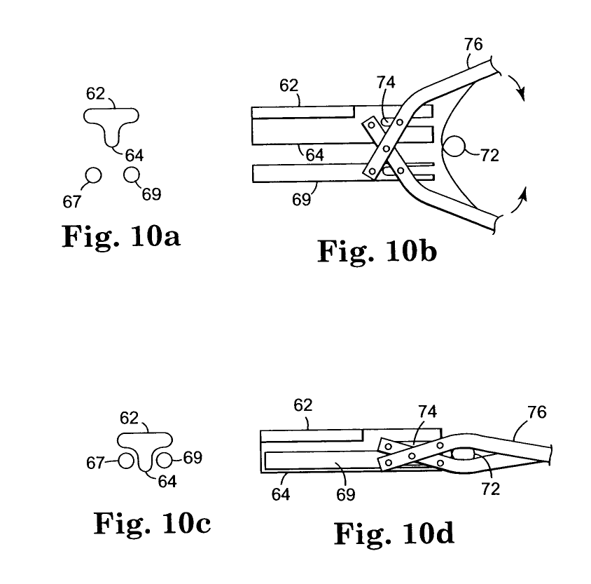

FIGS. 10a-10d show another embodiment of tension control article 60 according to the present invention. The tension control article 60 comprises a base portion 62 with integral tensioning member 64, handle 76, and movable tensioning members 67 and 69.

The tension control article 60 is movable between an open position (FIGS. 10c and 10d) with the tensioning members 67 and 69 spaced from base portion 62 so that the tension control article 60 may readily receive a sling, and a closed position (FIGS. 10a and 10b) with the tensioning members 67, 69 closer to the base portion 62 than in the open position.

A spring 72 biases the tension control article 60 toward the closed position. Manual pressure on handle 76 moves the tension control article from the closed toward the open position. The tension control article includes a hinge structure 74 that is preferably designed so that major surfaces of the base portion 62 and tensioning members 67, 69 remain substantially parallel during movement between the open and closed positions. Parallel movement between these structures is believed to avoid sling material extruding out of the open end of the tension control article 60 as the sling is being associated with the tension control article 60.

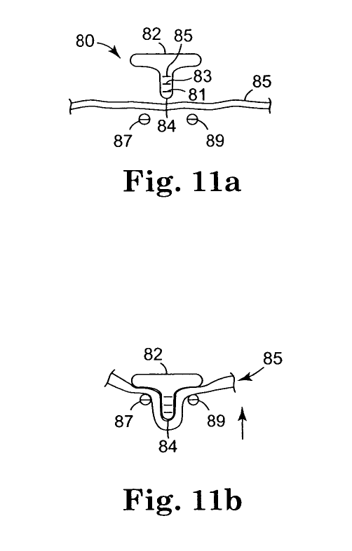

FIGS. 11a-11b show another embodiment of tension control article 80. The tension control article comprises a base portion 82 with integral tensioning member 84, a handle, and movable tensioning members 87 and 89. The tension control article 80 is movable between an open position (FIG. 11a) with the tensioning members 87 and 89 spaced from base portion 82 so that the tension control article 80 may readily receive a sling 85′, and a closed position (FIGS. 11b) with the tensioning members 87, 89 closer to the base portion 82 than in the open position.

The tension control article 80 includes tension level indicators 81, 83 and 85 on tensioning member 84. The indicators 81, 83 and 85 may comprise printing, molded in indicia or other forms of indicia. Members 87 and 89 may also include indicia thereon. The position of the indicators 81, 83 and 85 relative to the indicia on members 87 and 89 provide an indication of the tension provided by the tension control article 80.

Preferably, the tension control article 80 includes structure that releasably indexes the tension members 87, 89 between locations adjacent indicators 81, 83 and 85. A releasable detent and groove associated with a hinge provides suitable structure. Locations 81, 83 and 85 correspond to predetermined positions between the open (FIG. 11a) and fully closed (FIG. 11b) positions. Generally, the closer the tension members 87 and 89 are to the base portion 82, the more slack will be provided in sling 85′ when the tension control article 80 is removed.

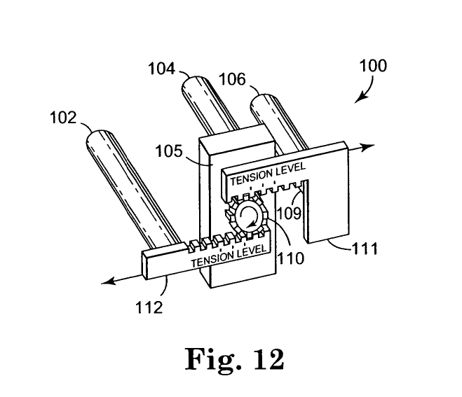

FIG. 12 illustrates another embodiment of tension control article 100. The tension control article 100 comprises tensioning members 102, 104 and 106, base member 105, and adjustment member 110. The tensioning members 102 and 106 are located on arms 112 and 111 that are movable relative to tensioning member 104. By rotating a geared wheel 110 that engages gears on arms 111 and 112, the tensioning members 102 and 106 may be moved away from or closer to tensioning member 104. Indicia 109 may be printed on or embossed on the arms 111 and 112 to provide an indication of the preselected tension provided by the tension control article 100. Adjustable tension control article 100 allows the surgeon to preselect a tension to account for the vagaries in human anatomy sizes, surgical procedure requirements or personal preference.

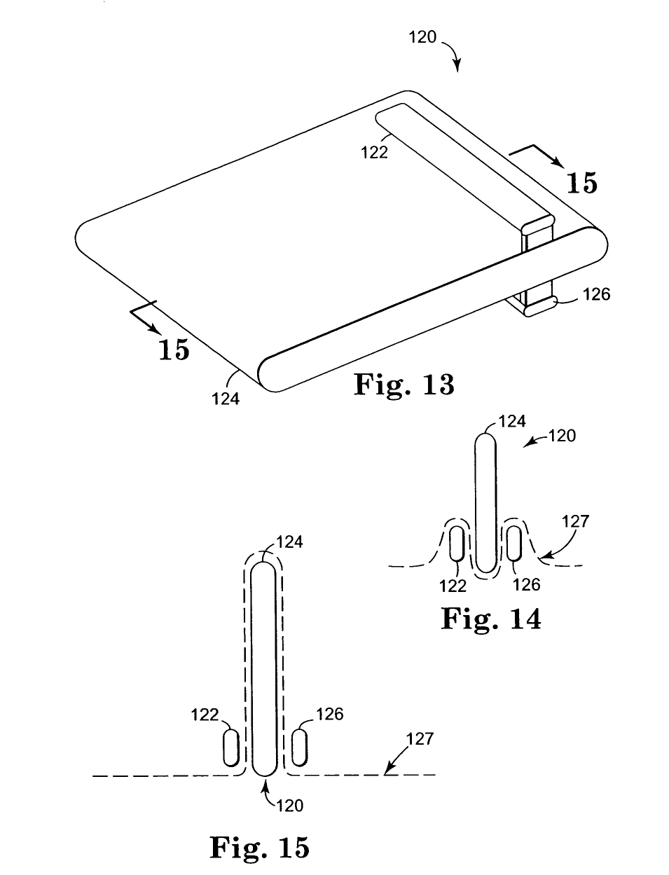

FIGS. 13-15 illustrate another embodiment of tension control article 120 according to the present invention. The tension control article 120 includes major tensioning member 124 and minor retention members 122 and 126. FIGS. 14 and 15 illustrate different tortuous paths associated with tension control article 120. Sling 127 is associated with article 120 by being threaded within tension control article 120 along one of the tortuous paths. The tortuous path of FIG. 14 is shorter than the tortuous path of FIG. 15. Generally, the longer the tortuous path, the more slack will be provided in the sling 127 when the tension control article 120 is removed. Also, the longer the tortuous path, the more slack is taken up in an implanted sling when the article 120 is applied to the sling.

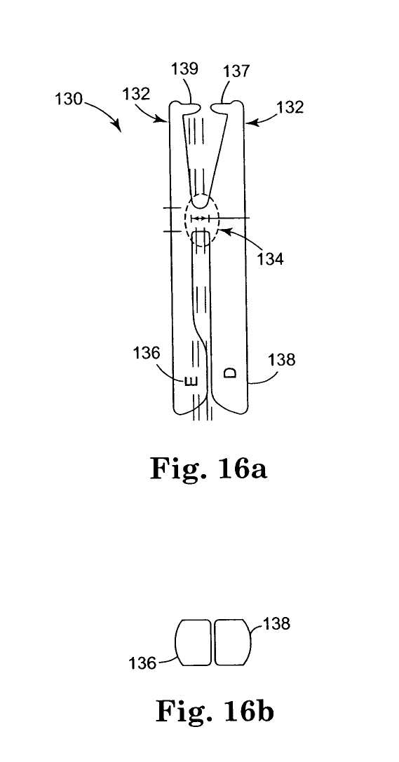

FIGS. 16a and 16b illustrate another embodiment of tension control article 130 according to the present invention. The tension control article 130 includes spacer jaws 136 and 138, hinge 134, handles 132 and over opening stops 137 and 139. The tension control article 130 is preferably a unitary structure with an inherent bias of the jaws toward a closed or clamped position.

The hinge 134 may comprise an integral or living hinge that biases the jaws 136 and 138 toward a closed position. In use, manual pressure is applied to handles 132 to open the jaws. A sling is placed in the open jaws and the handle is released. Upon release, the sling is clamped between the jaws 136 and 138. The sling is then placed next to the urethra with jaw 136 located between the sling and the urethra. The jaw 136 is sized and shaped to provide a predetermined distance between the urethra and sling. The tension control article 130 is then removed. The predetermined distance or size of jaw 136 assists the surgeon in providing a consistent, uniform and repeatable amount of looseness in a sling.

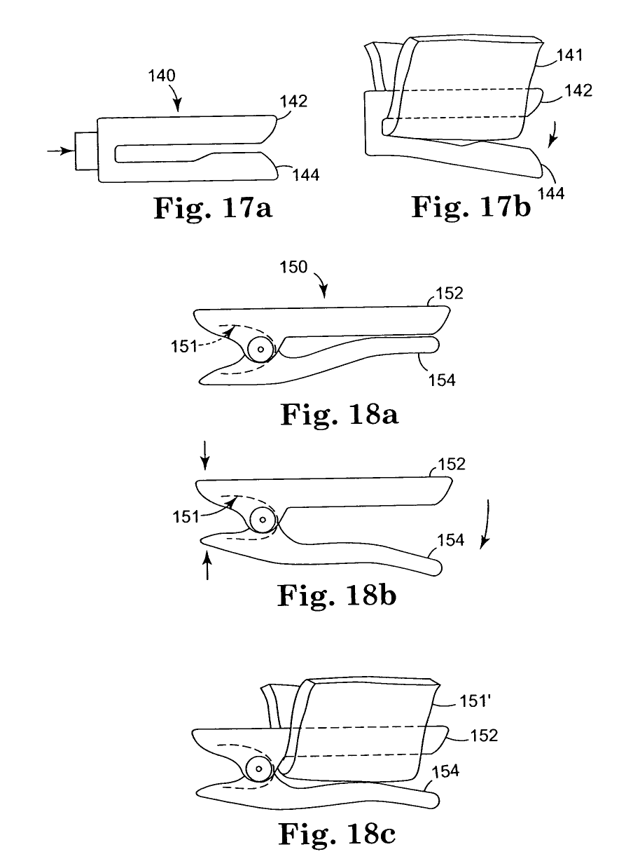

Another embodiment of tension control article 140 is shown in FIGS. 17a and 17b. Tension control article 140 includes jaws 142 and 144. A sling 141 is shown placed within the jaws in FIG. 17b. Optionally, jaw 142 may be constructed to be a different size than jaw 144 to afford two different spacing options for the surgeon.

FIGS. 18a through 18c show another embodiment of tension control article 150 according to the present invention. The tension control article 150 includes jaws 152 and 154, a hinge and a spring 151 for biasing jaws 152 and 154 toward a closed position. Sling 151′ is shown placed between jaws 152 and 154 in FIG. 18C.

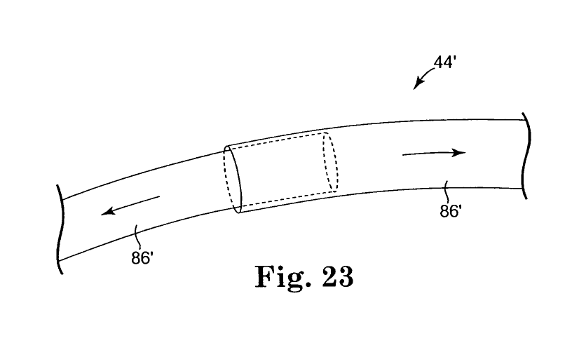

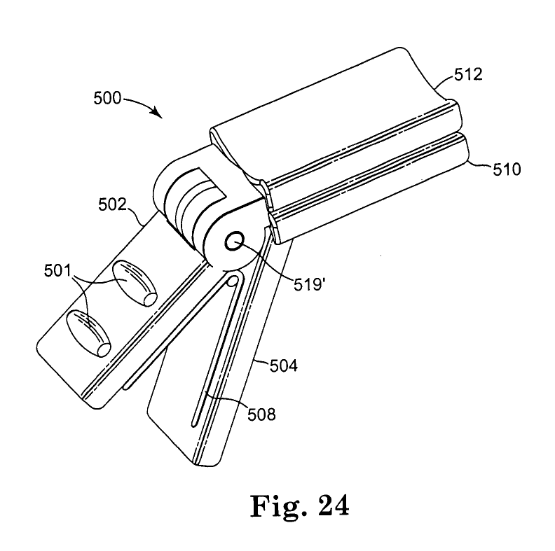

Sling assemblies for use in various aspects of the present invention may optionally include insertion sheaths. FIG. 23 shows components of a sling assembly 44′ that include overlapping sheath portions 86′. As stated above, when a sling assembly is placed in a U-shaped position underneath the patient's urethra, the portion of the sling assembly underneath the urethra (the portion of the U-shape with the greatest curvature) is preferably free of any sheath overlap.

FIGS. 24 through 34 show a variety of different surgical articles according to another aspect of the present invention. The surgical articles shown in these figures are particularly suitable for use with a sling assembly. A wide variety of different sling assemblies are contemplated for use with the instruments of FIGS. 24 through 34. The sling assemblies may comprise synthetic assemblies or composite sling assemblies. The slings may comprise synthetic materials or nonsynthetic materials (e.g. the materials described above). The sling assemblies may be implanted by a wide variety of surgical approaches such as transabdominal (i.e. suprapubic or from above), transvaginal (from below), or transobturator (e.g. with the sling anchored in the obturator foramen). Various surgical tools for implanting sling assemblies, sling assemblies and surgical approaches are disclosed in U.S. Pat. No. 6,612,977; published U.S. patent application Ser. Nos. 2002-0107430-A1, 2002-0147382, 2002-0099258-A1, 2003/0130670-A1 and U.S. Ser. No. 2002-0099259-A1; and U.S. patent application Ser. Nos. 10/306,179 filed Nov. 27, 2002 and 10/386,897, filed Mar. 11, 2003.

The surgical articles of FIGS. 24 through 34 are particularly suitable for use with a sling assembly comprising a synthetic sling with first and second end portions and a mid portion, and first and second insertion sheaths covering the first and second end portions. Preferably, the mid portion of the sling is substantially free of insertion sheath.

Referring now to a preferred embodiment shown in FIGS. 24 through 26, the present invention comprises a surgical article 500 for associating with a sling assembly (FIG.'s 25 and 26) with a sling 509 having first and second end portions and a mid portion, a first insertion sheath 542 substantially enclosing the first end portion of the sling, and a second insertion sheath 544 substantially enclosing the second end portion of the sling. The first and second insertion sheaths 542 and 544 having ends 543 and 545 that are spaced apart so that the mid portion of the sling 509 is free of any overlapping insertion sheath. The hiatus between the ends 543 and 545 helps ensure that the insertion sheaths 542 and 544 do not interfere with each other when being removed. The sling assembly is also free to form a support shape (e.g. a U-shape) without any overlapping of the insertion sheaths. The space between the ends 543 and 545 is preferably between about 0.05 inches and about 4 inches, more preferably between about 0.1 inch and about 1.5 inches.

The surgical article comprises a first jaw 512, and a second jaw 510. The jaws may comprise flat jaws, or alternatively, as shown in FIGS. 24-26, the jaws are preferably arcuate. The arcuate shape of the jaws is preferred when the surgical article is placed at approximately the mid portion of the sling as the arcuate shape better approximates the shape of the mid portion of the sling when it is placed in an anatomical support position. As examples not intended to be limiting, the jaws may have a radius of curvature in the range from about 0.2 inches to 1 inch (0.5 cm to 2.5 cm).

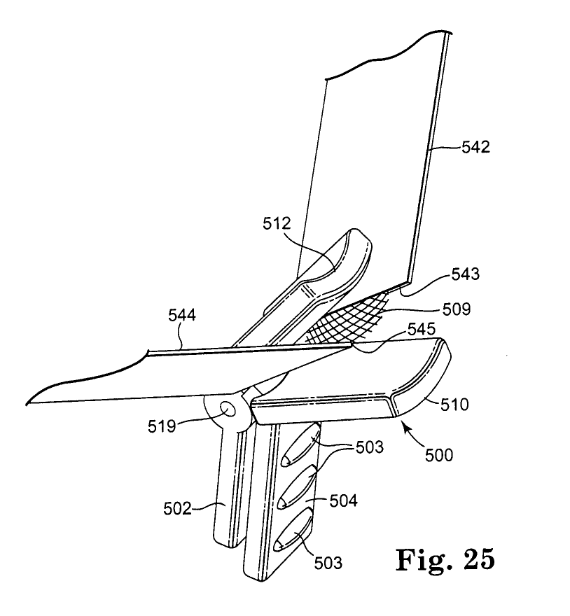

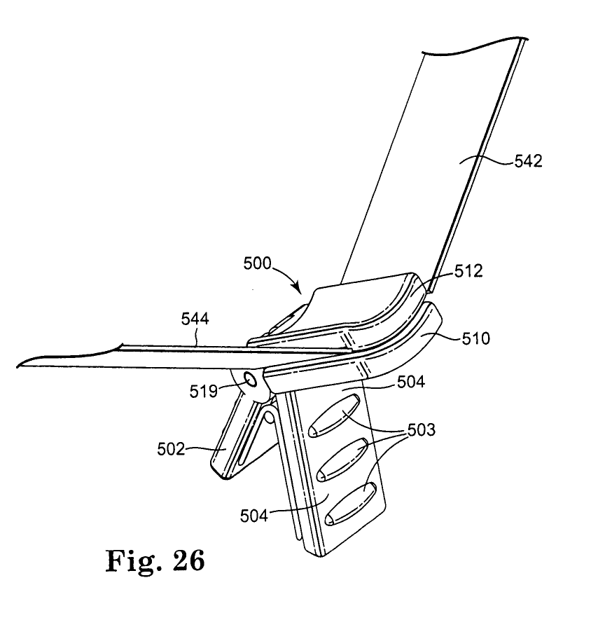

The first and second jaws 510, 512 are movable between an open position and a closed position. In the open position, the jaws 510, 512 receive the sling assembly or sling therebetween (FIG. 25). In the closed position (FIG. 26), the jaws associate the surgical article with the sling assembly or sling. A hinge rod 519′ may be conveniently utilized for this purpose.

As best seen in FIGS. 25 and 26, the first and second jaws preferably have a width (e.g. the length along the longitudinal axis of the sling). Preferably, the width is greater than the space between the ends 543 and 545 of the first and second insertion sheaths. In this preferred embodiment, the inner surfaces of the first and second jaws 510 and 512 engage the end regions of the first and second insertion sheaths 542 and 544.

In an alternative embodiment, the width of the jaws is less than the space between the ends 543 and 545 of the first and second insertion sheaths. This affords convenient removal of the insertions sheath 542 and 544 prior to removal of the surgical article 500.

In another embodiment, edge portions of the jaws may be received within the lumen of end portions of the first and second sheath 542 544 (e.g. near ends 543 and 545). This affords removal of the insertion sheaths 542, 544 independent of the operation of the surgical article 500. In this embodiment substantially all of the sling 509 is covered by either the insertion sheaths 542, 544 or the surgical article.

The surgical articles may be constructed from any suitable medical materials including, without limitation, silicone elastomer, acetate, acetal polyurethane, acrylic, elastomer, stainless steel, polysulfone, nylon, polycarbonate, polyethermide, acetal, ABS, bioresorbable materials or other biologically-compatible materials for temporary or permanent implantation. The materials can be rigid or conformable or combinations thereof. For example, if the inner surfaces of the jaws 510 and 512 are constructed of a conformable material, they can conform to the slight discontinuity between the sling 509 and sheaths 542 and 544 where the mid portion of the sling emerges from the ends of the insertion sheaths 542 and 544.

In one embodiment, the sling 509 comprises a mesh material. The mesh material may comprise one or more woven, knitted or inter-linked filaments or fibers that form multiple fiber junctions throughout the mesh. The filaments may comprise monofilaments or braided filament. The fiber junctions may be formed via weaving, knitting, braiding, bonding, ultrasonic welding or other junction forming techniques, including combinations thereof. In addition, the size of the resultant openings or pores of the mesh may be sufficient to allow tissue in-growth and fixation within surrounding tissue. As an example, not intended to be limiting, the holes may comprise polygonal shaped holes with diagonals of 0.132 inches and 0.076 inches.

The quantity and type of fiber junctions, fiber weave, pattern, and material type influence various sling properties or characteristics. As another example, not intended to be limiting, the mesh may be woven polypropylene monofilament, knitted with a warp tricot. The stitch count may be 27.5 courses/inch (+ or − 2 courses) and 13 wales/inch (+ or − 2 wales). The thickness of this example is 0.024 inches. Non-mesh and composite sling configurations are also included within the scope of the invention.

In one embodiment, a polypropylene sling mesh is constructed of polypropylene monofilament. The mesh may be precut to a predetermined size (e.g. about 1.1 cm width×35 cm length). An absorbable tensioning suture is preferably threaded into the length of the sling mesh to allow for tensioning adjustment of the sling mesh after placement in the patient is achieved.

In a preferred embodiment, the mesh 509 is preferably an elastic, as opposed to a substantially inelastic mesh. A test for differentiating between elastic meshes and substantially inelastic meshes is disclosed in U.S. patent application Ser. No. 10/386,897, filed Mar. 11, 2003 (the entire contents of which are herein incorporated by reference).

Preferably, one of the jaws (e.g. first jaw 512) has a predetermined size and shape for assisting a surgeon in placing proper tension on the sling. For example, the size and shape 512 of the first jaw may approximate the structure of the Mayo scissors, Hegar clamp or other device recommended in the Instructions for Use for the TVT Sling procedure (from Ethicon) or the SPARC or Monarc surgical procedures (available from American Medical Systems, Inc. of Minnetonka, Minn.), or the various journal articles cited herein. The desired depth of the jaw can depend on a variety of factors including the type of instrument it is designed to mimic, the width and length of the jaw, and the desired tension or slack desired. As an example not intended to be limiting, the first jaw may have a depth of between about 0.5 mm and about 10 cm, more preferably, the depth is between about 1 mm and about 2.5 cm.

The first and second jaws 510 and 512 have sling assembly engagement surfaces. As described above, the sling assembly engagement surfaces are preferably adapted to engage the first and second insertion sheaths of the sling assembly. Alternatively, the sling assembly engagement surfaces may be adapted to engage the mid portion of the sling solely or a combination of the insertion sheaths and sling 509.

In one embodiment, the surgical article is sized and shaped to substantially enclose the mid-portion of the sling.

The surgical article preferably includes a biasing means 508 for biasing the first and second jaws 512 and 510 toward the closed position. Any suitable biasing means may be utilized including, for example, a coil spring, a leaf spring or a resilient material with an inherent bias.

The surgical article preferably includes manually engageable handles 502 and 504. The handles 504 and 502 are operatively associated with the first and second jaws 512 and 510 for manually moving the jaws from the closed position toward the open position. Preferably, the handles 502 and 504 include grasping ribs 501, 503.

In another preferred embodiment, the handle 502 is fixed and cannot move relative to jaw 512.

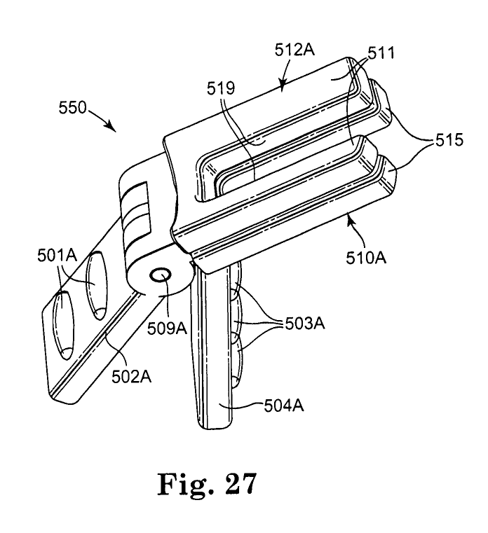

Referring now to FIG. 27, there is shown another embodiment of surgical article 550 according to the present invention. The surgical article 550 includes components similar to the components of the surgical article 500 to which the suffix A has been added. Like the article 500, the article 550 may include a hinge rod 509A. Unlike the surgical article 500, the surgical article 550 includes a slot 519 in the jaws 510A and 512A. The slot 519 may be formed by fingers 511, 515 of jaws 510A and 512A.

The slot 519 affords access to the sling assembly and sling. The access may be for visual inspection or other purposes. For example, the slot 519 affords the opportunity for the surgeon to apply a therapeutic agent (e.g. an antibiotic or antimicrobial) to the sling or sling assembly. The slot 519 may also be sized and shaped to afford a suture throw through the sling or sling assembly.

In another embodiment, the slot may be sized and shaped to accommodate a bulking means/device. In this embodiment, the bulking means are placed between the sling and the urethra while including a jaw 511 that may be sized and shaped to afford proper tension of the sling.

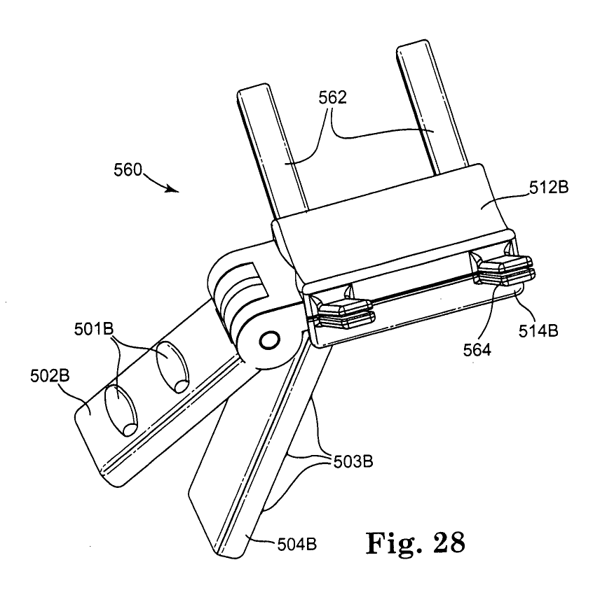

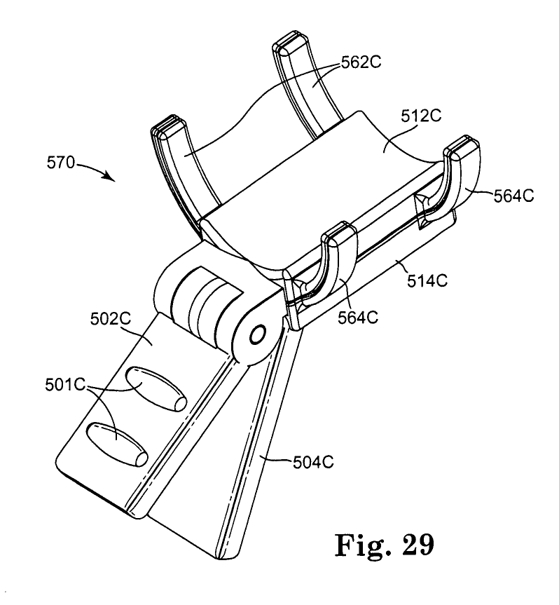

Referring now to FIG. 28, there is shown another embodiment of surgical article 560 according to the present invention. The surgical article 560 has many of the same components of the surgical article 500 to which the suffix B has been added.

The article 560 includes arms 562 and 564 extending from the jaws 512B and 514B. The article 560 may be used to control the shape of the sling, sheaths, sling assembly or portions thereof. For example, the article 560 may be used to reduce the amount of the sheath placed in the bottom of an intended U-shaped configuration. This is believed to reduce the difficulty in removing the insertion sheaths during implantation. The arms 562 and 564 may be constructed to be sufficiently flexible to allow them to deflect during implantation of the sling and attached tool to follow the path of the implanted sling.

The arms 562 and 564 may be molded so that they assume a predetermined form that would closely mimic the placement of a sling via a predetermined surgical approach (e.g. suprapubic or transobturator). Preferably the flexibility is sufficient to afford variations in procedures and anatomies.

Referring now to FIG. 29, there is shown another embodiment of surgical article 570 according to the present invention. The surgical article 570 has many of the same components of the surgical article 500 to which the suffix C has been added. In this embodiment, the arms 562C and 564C are substantially arcuate to more closely mimic placement of a sling in a U-shaped configuration.

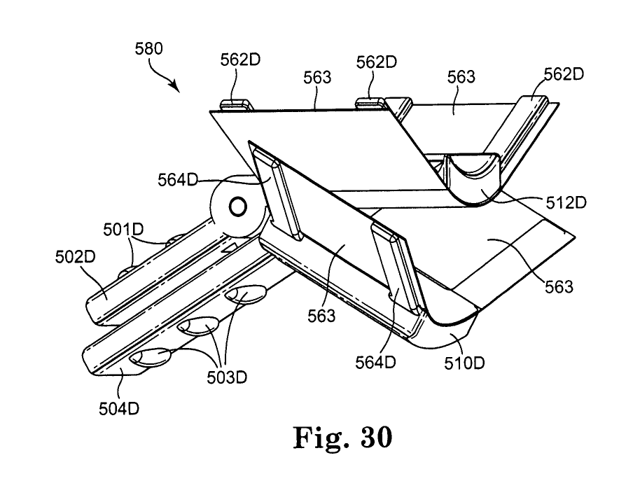

Referring now to FIGS. 30, 31 and 33, there is shown another embodiment of surgical article 580 according to the present invention. The surgical article 580 has many of the same components of the surgical article 500 to which the suffix D has been added. The surgical article 580 also has many of the components of the surgical article 560 (FIG. 28).

The surgical article 580 includes thin material portions (e.g. webs) 563 that are attached to the arms 562D and 564D (e.g. by being integrally molded therewith, molded in a second molding process, or bonded or otherwise affixed). The surgical article 580 more completely covers the sling and/or sheath(s) during implantation than the article 560. The material of the portions 563 may be the same as the material of surgical article 580 or it may be a different material (e.g. the material of the sheath).

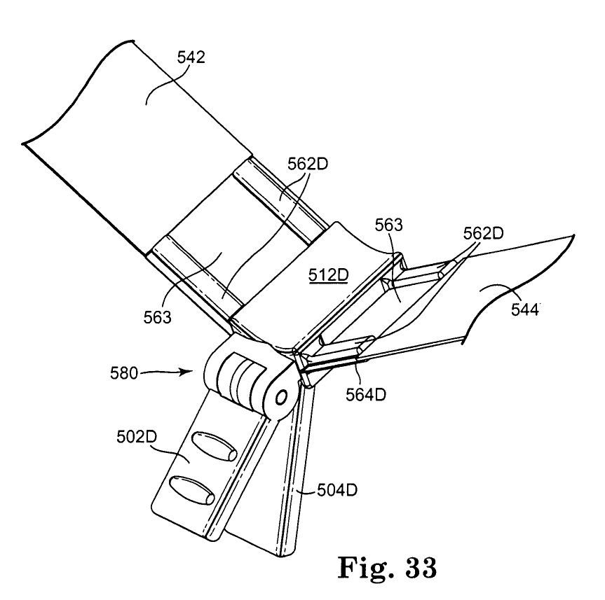

In another embodiment, unlike the showing in FIG. 33, the end portions of the insertion sheaths 542 and 544 may extend over the arms 562D and 564D so that the arms are telescopically received within a lumen of the end portions of the insertion sheaths 542 and 544.

In another embodiment the arms 562D and 564D may be removed or absent affording just the thin wafer of material 563 extending from the jaws 510D and 512D. The web can be designed to flex during implantation, providing a greater area of coverage and reducing the difficulty in removing the implantation sheaths.

The surgical article 580 is particularly suitable for use with a sling assembly that is sized and shaped to be implanted using a transobturator approach. However, the wafer may be constructed to be flexible so that the same surgical article 580 may be used with a sling assembly that is sized and shaped for implantation during a suprapubic approach.

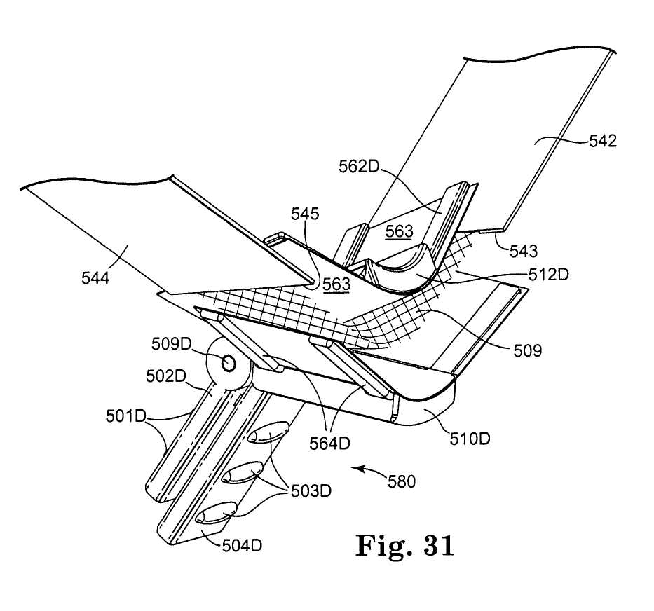

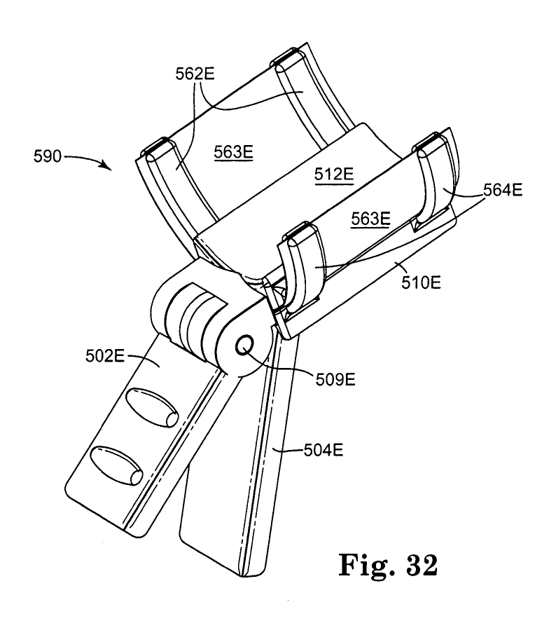

FIG. 33 is another perspective view of the components of FIG. 31 with the jaws of the surgical article in a closed position. In alternative embodiments, the surgical article 580 may be designed to engage the insertion sheaths 542, 544, the sling 509 or both.

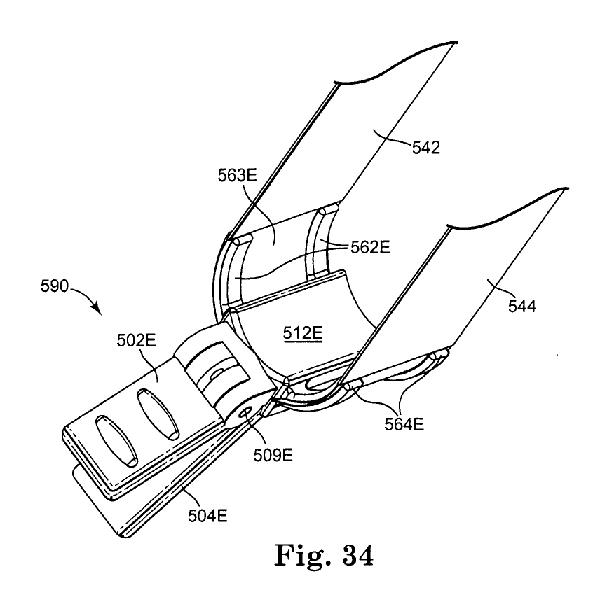

Referring now to FIGS. 32 and 34, there is shown another embodiment of surgical article 590 according to the present invention. FIG. 34 is another perspective view of the surgical article of FIG. 32 with jaws in a closed position and a portion of a sling assembly received therebetween. The surgical article 590 has many of the same components of the surgical article 500 to which the suffix E has been added.

The surgical article 590 is particularly suitable for use in suprapubic surgical approach where the sling is intended to be placed in a U-shaped configuration.

The device according to the invention may be easily tailored to provide increased or decreased urethral support. Optionally, the tension control article may allow the surgeon to remove, or break the tensioning members during the surgical procedure or during a post operative period.

### Method

In another aspect, the present invention comprises a method of changing tension of an implantable article using an article according to the present invention. The article may be implanted in the body during a surgical procedure. Alternatively it may be removed prior to the end of a surgical procedure. The article of the present invention may be modified in a subsequent surgical procedure or by substantially non-invasive means.

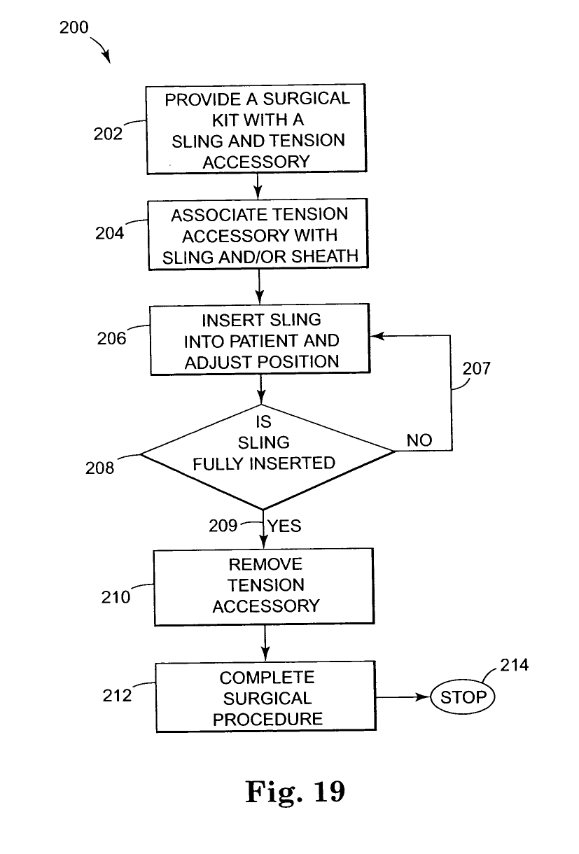

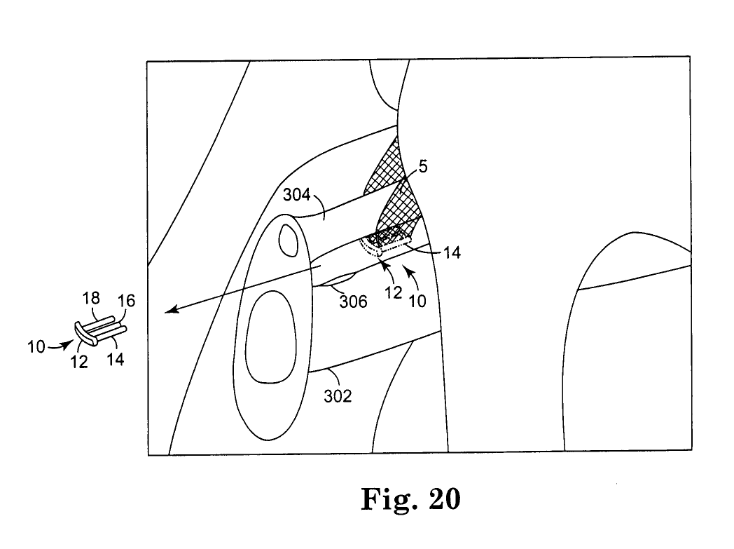

FIGS. 19 and 20 illustrate a method where the surgical article is removed prior to the end of the surgical procedure. FIG. 19 is a flowchart illustrating an embodiment of method 200 according to the present invention.

Step 202 provides a tension control article (tension accessory) and a sling. Preferably, the tension control article (e.g. 10) may be part of a surgical kit. The kit may be a surgical kit having tools for treating incontinence, such as a sling kit. Alternatively, the tension control article, sling and the rest of the surgical articles may be independently provided to the surgeon. The latter case is desirable when the elements of the kit have drastically different shelf lives or storage condition requirements (e.g. refrigeration).

Step 204 associates the tension accessory (the tension control article) with the sling. Optionally, the manufacturer can conduct this step so that a sling/tension control article preassembly is provided to the surgeon in the kit. Alternatively, this step may be conducted by the surgeon or other healthcare professional prior to implanting the sling, especially if the tension control article provides a plurality of different tension options.

Using tension control article 10 as an example, to associate the article 10 with a sling, the operator threads the sling along one of the tortuous paths provided by the article 10. It is noted that, if the sling includes a surrounding, removable plastic sheath, the sling/sheath combination may be threaded along the tortuous path. Optionally, the sling/sheath combination may include a hiatus (e.g. a portion with no plastic sheath) at about the mid portion of the sling to allow the sling to be threaded between the tensioning members of the article 10 without interference from the sheath.

In step 206, the sling is inserted in the body and adjusted to a predetermined position. For example, some sling procedures call for a tension free sling. For such a procedure, the sling/tension control article combination can be situated in a fully inserted position such that the sling and/or the tension control article are just adjacent or even slightly touching the urethra.

FIG. 20 schematically illustrates article 10 in a fully inserted position with solid lines. The article 10 is just adjacent urethra 304. Vaginal incision 306, vagina 302, and sling S are also shown. In step 208 (FIG. 19), the surgeon verifies that the sling/tension control article combination are in this fully inserted position. If not, the surgeon continues to adjust 207 the sling. If the combination is fully inserted, then the surgeon verifies the fully inserted position 209. Once the fully inserted position is verified, the surgeon may remove the tension control article in step 210. This removal step is illustrated with an arrow and dotted lines in FIG. 20. Step 210 may be conducted before or after any optional insertion sheath is removed from the sling, but it preferably occurs after any such optional insertion sheath is removed.

Changing the tension of the sling S at a location substantially adjacent the urethra 304 is more effective than attempting to modify the tension of a sling at a location remote from the urethra. This is particularly the case where an axially extendable synthetic sling (a polypropylene sling with holes) and insertion sheath are used as, once the insertion sheath is removed, adjustment of the entire length of the sling is particularly difficult or problematic due to the interaction between tissue and the sling.

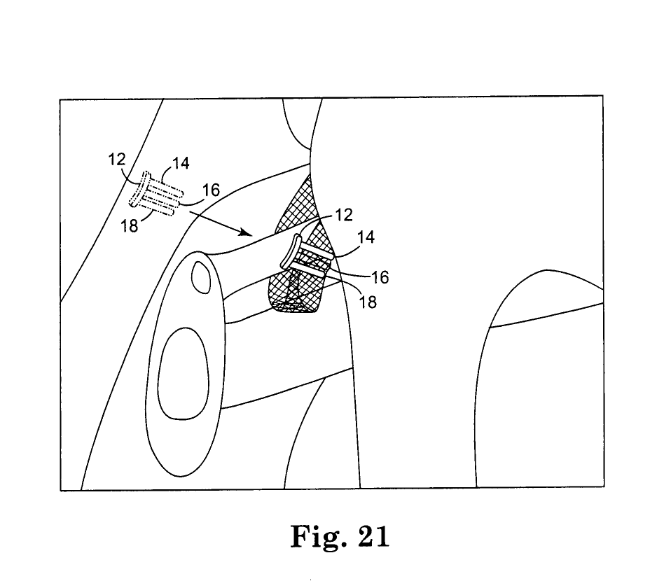

FIG. 21 illustrates another embodiment of method according to the present invention. In this embodiment, the sling is excessively loose. Article 10 is placed on the sling (see the arrow and solid lines) to take up excessive slack in the sling. In this instance, article 10 is preferably left in the body after the surgical procedure. Preferably, by positioning the sling within the tortuous pathway formed by the plurality of tensioning members, article 10 effectively increases the path length traversed by the support material, thereby resulting in a reduction or elimination of slack from the support material and an increase in supportive tension unless and until the tension control article is removed. As shown in FIGS. 2a and 2b, multiple degrees of retentive force may be applied to anatomical support material by a device 10. For example, FIG. 2a shows one method of practice wherein the device 10 provides a tortuous pathway for a section of anatomical support material 20 disposed thereon. An alternative method of practice is shown in FIG. 2b, wherein an alternate tortuous pathway is created. FIG. 12 illustrates an article with a feature that allows the surgeon to adjust the tension provided by the clip. In preferred embodiments, the present invention permits the user to vary the supportive tension applied to the anatomical support material disposed therein by altering the tortuous pathway traversed by the anatomical support material.

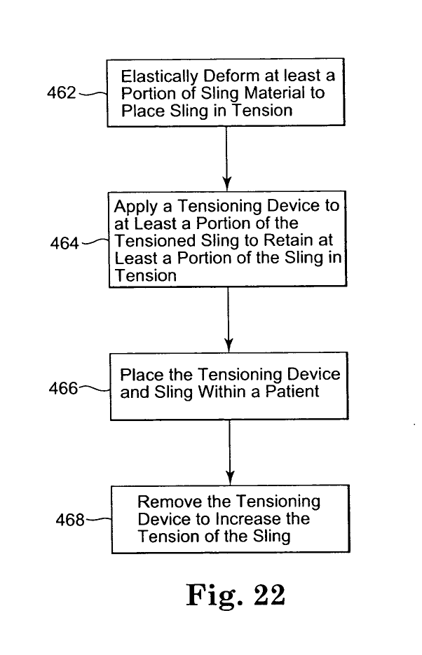

It is also noted that removal of an article from its association with the sling can be exploited to increase the tension of the sling on the urethra. FIG. 22 is a flow chart illustrating a method of increasing the tension of a sling by removing a tension control article from its association with the sling.

FIG. 22 illustrates a method of treating incontinence comprising the steps of: (i) providing a support material with at least a portion that is elastically deformable, (ii) elastically deforming at least a portion of the support material to tension the support material (see step 462); (iii) providing a tensioning article, (iv) applying the tensioning article to the tensioned support material to retain at least a portion of the support material in an elastically deformed condition (see step 464); (v) implanting the support material with applied tensioning article in a patient (466); and vii) removing the tensioning article to increase the tension provided by the support material 468. The tension control article used in this method is preferably one with a locking member (FIG. 5a or 5b) or one that can clamp the sling between jaw members (e.g. FIGS. 10a-11b) so that the elastic deformation can be held in the sling material.

The tension control article may be provided in a kit or provided independent of other surgical articles. One or more articles may be used during a surgical procedure. The tension control article may be positioned on a portion of anatomical support material at the time of manufacture, immediately prior to, or following the surgical implantation of the anatomical support. For example, one surgical urethral stabilization procedure comprises attaching anchors to an internal structure, for example, the posterior or inferior pubic bone, and affixing a mesh sling to said anchors, thereby resulting in supportive force being applied to the urethra. The tension control article 10 may be applied to the anatomical support material according to FIG. 2a or 2b should the anatomical support fail to provide adequate support to the anatomical structure. Applying the tensioning device 10 to the anatomical support material increases the pathway between the attaching anchors traversed by the anatomical support and increases the support tension applied by the device. The practitioner may easily vary the amount of supportive tension by applying more or fewer anatomical support tension control articles. Alternatively, the amount of supportive tension applied by the anatomical support tensioning device may be varied by applying tensioning device having more or fewer tensioning members disposed thereon.

In another aspect, the present invention involves the use of the surgical articles of FIGS. 24-34 in a surgical sling procedure. For example, the surgical article 500 may be placed about the mid portion of the sling. This does away with the overlapping sheath portions. The sling assembly can be first implanted using known surgical approaches (e.g. suprapubic and transobturator) and then the surgical article 500 can be removed. With no overlapping portions underneath the urethra (i.e. in a position of maximum curvature), the insertion sheaths 542 and 544 are more easily and conveniently removed. Also, the amount of exposed insertion sheath in the region of the vaginal incision is also reduced. This reduces the amount of insertion sheath material that is moved from the region of the vaginal incision to a skin incision (e.g. in the abdomen or adjacent the obturator foramen). This is believed to reduce the chances that a contaminant from the vaginal region is transported to another region of the body.

The present invention contemplates methods where the insertion sheaths 542 and 544 are initially removed and then the surgical article 500 is removed. This allows the sling to be inserted and the insertion sheaths removed without disturbing the article 500 (and the desired distance between the top jaw and the urethra). Alternatively, the article 500 can be initially removed and then the insertion sheaths 542 and 544 could thereafter be removed. The present invention optionally includes embodiments that utilize a tensioning suture to fine tune the final placement of the sling.

All patents, patent applications, and publications cited herein are hereby incorporated by reference in their entirety as if individually incorporated.

It is understood that the embodiments of the invention disclosed herein are illustrative of the principles of the invention. Other modifications may be employed which are within the scope of the invention; thus, by way of example but not of limitation, alternate base member shapes, alternative tensioning member shapes, and use with alternative anatomical support materials. Accordingly, the present invention is not limited to that precisely as shown and described in the present invention.

What is claimed is:

1. A surgical article for associating with a sling assembly comprising a sling having first and second end portions and a mid portion, a first insertion sheath substantially enclosing the first end portion of the sling, a second insertion sheath substantially enclosing the second end portion of the sling, the first and second insertion sheaths having ends that are spaced apart so that the mid portion of the sling is free of any insertion sheath, the surgical article comprising:
a first jaw,
a second jaw,
the first and second jaws being movable between an open position to receiving the sling assembly therebetween, and a closed position for associating the surgical article with the sling assembly; and
wherein the first jaw has a predetermined size and shape for assisting a surgeon in implanting a sling with appropriate tension.

2. An article according to claim 1 wherein the first jaw has a width and the width is greater than the space between the ends of the first and second insertion sheaths.

3. An article according to claim 1 wherein the first and second jaws are arcuate shaped.

4. An article according to claim 1 wherein the first jaw has a depth of between about 0.5 mm and about 10 cm.

5. An article according to claim 1 wherein the first and second jaws have sling assembly engagement surfaces.

6. An article according to claim 5 wherein the sling assembly engagement surfaces are adapted to engage the first and second insertion sheaths of the sling assembly.

7. An article according to claim 5 wherein the sling assembly engagement surfaces are adapted to engage the mid portion of the sling without engaging any portion of the first and second insertion sheaths so that the insertion sheaths may be removed without removing the surgical article.

8. An article according to claim 1 wherein the surgical article is sized and shaped to substantially enclose the mid-portion of the sling.

9. An article according to claim 1 further including biasing means for biasing the first and second jaws toward the closed position.

10. An article according to claim 1 further including manually engageable handles, operatively associated with the first and second jaws, for manually moving the jaws from the closed position toward the open position.

11. An article according to claim 1 wherein the first or second jaw includes a slot.

12. An article according to claim 1 wherein the first and second jaws include appendages projecting therefrom to hold portions of the sling assembly in a predetermined orientation.

13. An article according to claim 12 wherein the appendages are flexible so that they can deflect.

14. An article according to claim 1 wherein the first and second jaws include appendages projecting therefrom to hold a portion of the sling in a predetermined orientation.

15. An article according to claim 1 wherein the first and second jaws include appendages projecting therefrom, and
a thin material situated between the appendages projecting from a jaw.

16. An article according to claim 15 wherein jaws, appendages and thin materials are adapted to substantially engage the mid-portion of the sling.

17. An article according to claim 1 wherein the jaws are shaped to approximate the intended shape of the mid portion of the sling after implantation via a transobturator surgical approach.

18. An article according to claim 1 wherein the jaws are shaped to approximate the intended shape of the mid portion of the sling after implantation via a suprapubic surgical approach.

19. In combination, a sling assembly comprising a sling having first and second end portions and a mid portion, a first insertion sheath substantially enclosing the first end portion of the sling, a second insertion sheath substantially enclosing the second end portion of the sling, the first and second insertion sheaths having ends that are spaced apart so that the mid portion of the sling is free of any insertion sheath,
a surgical article comprising a first jaw, a second jaw, the first and second jaws being movable between an open position to receiving the sling assembly therebetween, and a closed position for associating the surgical article with the sling assembly; and
wherein the first jaw has a predetermined size and shape for assisting a surgeon in implanting a sling with appropriate tension.

20. A combination according to claim 19 wherein the sling comprises a polypropylene material and the insertion sheaths comprise a polyethylene material.

21. A method of treating a patient's urological disorder comprising the steps of inserting a sling assembly comprising a sling with a mid-portion, the sling having insertion sheaths enclosing end portions thereof, the insertion sheaths having ends proximate the mid-portion of the sling that are spaced apart, and a spacer article associated with the sling assembly;
verifying that the spacer article is in at least close proximity to the patient's urethra;
removing the spacer article; and
removing the insertion sheaths.

22. A method according to claim 21 wherein the step of removing the spacer occurs before the step of removing the insertion sheaths.

23. A method according to claim 21 wherein the step of inserting the sling assembly includes the step of making a vaginal incision, and
the step of removing the spacer article includes the step of removing the spacer article through the vaginal incision.

24. A method according to claim 21 wherein the step of inserting the sling assembly includes the step of using a transobturator surgical approach.

25. A method according to claim 21 wherein the step of inserting the sling assembly includes the step of using a suprapubic surgical approach.

26. A method according to claim 21 the step of removing the insertion sheaths occurs before the step of removing the spacer.
